(19) United States
(12) Patent Application Publication
(10) Pub. No.: US 2024/0002829 A1
(43) Pub. Date: Jan. 4, 2024

(54) IMMOBILIZED CRUDE ENZYME FOR DEGRADING COMPLEX POLYCYCLIC AROMATIC HYDROCARBONs (PAHs) IN SOIL AND PREPARATION METHOD THEREOF

(71) Applicant: Beijing Univ Civil Engn & Architecture, Beijing (CN)

(72) Inventors: Dawen GAO, Beijing (CN); Huan ZHAO, Beijing (CN); Hong LIANG, Beijing (CN)

(21) Appl. No.: 18/216,837

(22) Filed: Jun. 30, 2023

(30) Foreign Application Priority Data
Jul. 3, 2022 (CN) 202210776864.7

(51) Int. Cl. C12N 11/06; C12N 9/02; B09C 1/10

(52) U.S. Cl. CPC C12N 11/06; C12N 9/0061; C12Y 110/03002; B09C 1/105

(57) ABSTRACT

The present disclosure provides an immobilized crude enzyme for degrading complex polycyclic aromatic hydrocarbons (PAHs) in soil and a preparation method thereof, and relates to the field of remediation of complex organics-polluted soil. The present disclosure particularly relates to an immobilized crude enzyme and a preparation method thereof. In the present disclosure, the immobilized crude enzyme for degrading complex PAHs in soil is prepared from an acid-modified chestnut inner shell and a crude enzyme solution of white rot fungi; and there are copper ions in the crude enzyme solution of the white rot fungi. The preparation method includes: 1, preparing a chestnut inner shell into a powdered material; 2, preparing modified biochar; 3, conducting immobilization.

### CROSS REFERENCE TO RELATED APPLICATION

This patent application claims the benefit and priority of Chinese Patent Application No. 202210776864.7, filed with the China National Intellectual Property Administration on Jul. 3, 2022, the disclosure of which is incorporated by reference herein in its entirety as part of the present application.

### TECHNICAL FIELD

The present disclosure relates to the field of remediation of complex organics-polluted soil, and particularly relates to an immobilized crude enzyme and a preparation method thereof.

### BACKGROUND

The “National Soil Pollution Survey Bulletin” released in 2014 shows that the overall situation of soil environment is not optimistic. As the natural foundation of urban construction, urban soil is crucial to the survival and development of human society. However, with the relocation of chemical companies associated with petroleum and coal out of cities, soil pollution in legacy lands with polycyclic aromatic hydrocarbons (PAHs) as a main pollutant has become more serious, causing a series of urban ecological environmental problems.

Compared with the degradation of PAHs by microorganisms such as bacteria and fungi, the enzyme degrades PAHs more effectively, and can exert a desirable degradation effect on PAHs even under complex soil environmental conditions. Therefore, the remediation of PAHs-contaminated soil with enzymes is considered to be the most potential remediation method in the whole bioremediation. Most of the current researches focus on separation and purification of laccase in the crude enzyme solution of white rot fungi. The pure laccase after fractional precipitation and chromatographic purification is applied to the degradation of PAHs. However, purified enzyme preparations generally have a high cost. Moreover, neither the enzyme preparation nor the crude enzyme solution shows an ideal degradation effect on PAHs in the soil environment during actual applications.

### SUMMARY

In order to be applied in actual soil remediation and to achieve an ideal PAHs degradation effect, the present disclosure provides an immobilized crude enzyme for degrading complex PAHs in soil.

In the present disclosure, the immobilized crude enzyme for degrading complex PAHs in soil is prepared from an acid-modified chestnut inner shell and a crude enzyme solution of white rot fungi; and there are copper ions in the crude enzyme solution of the white rot fungi.

Further, a concentration of the copper ions in the crude enzyme solution of the white rot fungi is 1 mM to 2 mM.

Further, the crude enzyme solution of the white rot fungi further contains acetonitrile, Tween 80, and a mediator; the mediator contains 1-hydroxybenzotriazole (HBT) and violuric acid; and the crude enzyme solution has the HBT at a concentration of 0.1 mM to 0.5 mM, the violuric acid at a concentration of 0.5 mM to 1 mM, the acetonitrile at a concentration of 10%, and the Tween 80 at a concentration of 1%.

In an example of the present disclosure, the acetonitrile is added as a cosolvent to the crude enzyme solution of the white rot fungi, and the Tween 80 is added as a dispersant to the crude enzyme solution of the white rot fungi.

Further, the crude enzyme solution of the white rot fungi shows a laccase activity of U/mL.

Further, the crude enzyme solution of the white rot fungi has a natural pH value.

Further, the crude enzyme solution of the white rot fungi is prepared by subjecting a Coriolus versicolor strain to culture in a broth at 25° C. to 31° C. and 120 r/min to 160 r/min for 12 d to 18 d, conducting centrifugal separation at 9,000 r/min to 11,000 r/min for 10 mM to obtain a supernatant, and then diluting the supernatant; and 1 L of the broth contains 15.00 g to 30.00 g of bran, 0.40 g to 0.50 g of NH₄Cl, 0.20 g of KH₂PO₄, 0.05 g of MgSO₄·7H₂O, g of CaCl₂, 1.00 g of the Tween 80, 1.00 mL of an inorganic solution, and distilled water as a balance.

In an example of the present disclosure, the broth has a natural pH value.

In an example of the present disclosure, 1 L of the inorganic solution contains: 3.00 g of MgSO₄·7H₂O, 0.50 g of MnSO₄·H₂O, 1.00 g of NaCl, 0.10 g of FeSO₄·7H₂O, 0.10 g of CoCl₂, 0.10 g of ZnSO₄·7H₂O, 0.10 g of CuSO₄·5H₂O, 0.01 g of KAl(SO₄)₂·12H₂O, 0.01 g of H₃BO₃, 0.01 g of Na₂MoO₄·2H₂O, and distilled water as a balance.

The present disclosure further provides a preparation method of an immobilized crude enzyme for degrading complex PAHs in soil, including the following steps:
step 1, washing and drying a chestnut inner shell, and grinding into a powdered material;
step 2, subjecting the powdered material to pyrolysis in a crucible at 600° C. for 3 h, taking out after cooling, adding with a citric acid solution, and conducting modification by shaking in a shaker at 25° C. to 30° C. and 130 r/min to 180 r/min for 12 h to 48 h to obtain modified biochar; and
step 3, adding the modified biochar to the crude enzyme solution of the white rot fungi, shaking an obtained mixture in a shaker at 25° C. to 30° C. and 130 r/min to 180 r/min for 3 h to 48 h, conducting centrifugation at 5,000 r/min to 10,000 r/min for 5 min to 10 min, and collecting treated biochar to obtain the immobilized crude enzyme for degrading complex PAHs in soil.

In the present disclosure, the immobilized crude enzyme for degrading complex PAHs in soil uses an extracellular crude enzyme of the white rot fungi as a bioremediation material. The extracellular crude enzyme of the white rot fungi contains various oxidoreductases including laccase, which have a wider range of action on substrates, and also contain various natural mediator components. The immobilized crude enzyme for degrading complex PAHs in soil uses the acid-modified chestnut inner shell as a solid carrier. The acid-modified chestnut inner shell has a clear porous structure, which is beneficial to the loading, immobilization, and protection of the crude enzyme solution of the white rot fungi. In this way, the degradation effect of the crude enzyme of the white rot fungi on PAHs is greatly improved in an actual soil remediation environment.

### BRIEF DESCRIPTION OF THE DRAWINGS

FIG. 1 shows the changes of laccase activity in a crude enzyme solution during 35 d of culture on a strain in Specific Example VI in a broth at 25° C. to 31° C. and 120 r/min to 160 r/min;

FIGS. 2A-C show microstructure of modified biochar derived from a chestnut inner shell in Example 1, where FIG. 2A is ×350 magnification, FIG. 2B is ×500 magnification, and FIG. 2C is ×1100 magnification;

FIGS. 3A-C show microstructure of modified biochar derived from a chestnut outer shell in Example 1, where FIG. 3A is ×350 magnification, FIG. 3B is ×500 magnification, and FIG. 3C is ×1100 magnification;

FIGS. 4A-B show microstructure of modified biochar in Specific Example IX, where FIG. 4A is ×500 magnification, and FIG. 4B is ×800 magnification;

FIGS. 5A-B show microstructure of modified biochar derived from an acid-modified chestnut outer shell in Example 1, where FIG. 5A is ×500 magnification, and FIG. 5B is ×800 magnification;

FIGS. 6A-B show microstructure of modified biochar derived from an alkali-modified chestnut inner shell in Example 1, where FIG. 6A is ×500 magnification, and FIG. 6B is ×800 magnification;

FIGS. 7A-B show microstructure of modified biochar derived from an alkali-modified chestnut outer shell in Example 1, where FIG. 7A is ×500 magnification, and FIG. 7B is ×800 magnification;

FIGS. 8A-B show microstructure of rice husk-derived biochar in Example 1, where FIG. 8A is ×500 magnification, and FIG. 8B is ×800 magnification;

FIGS. 9A-B show microstructure of corn straw-derived biochar in Example 1, where FIG. 9A is ×500 magnification, and FIG. 9B is ×800 magnification; and

FIG. 10 shows the results of bioremediation experiment on PAHs-contaminated soil in Example 1.

### DETAILED DESCRIPTION OF THE EMBODIMENTS

The technical solutions of the embodiments of the present disclosure are clearly and completely described below with reference to the drawings. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

It should be noted that the examples in the present disclosure or features in the examples may be combined in a non-conflicting manner.
Specific Example I: In this specific example, the immobilized crude enzyme for degrading complex PAHs in soil is prepared from an acid-modified chestnut inner shell and a crude enzyme solution of white rot fungi; and there are copper ions in the crude enzyme solution of the white rot fungi.
Specific Example II: This specific example differs from Specific Example I only in that: a concentration of the copper ions in the crude enzyme solution of the white rot fungi is 1 mM to 2 mM.
Specific Example III: This specific example differs from Specific Example I or II only in that: the crude enzyme solution of the white rot fungi further contains acetonitrile, Tween 80, and a mediator; the mediator contains 1-hydroxybenzotriazole (HBT) and violuric acid; and the crude enzyme solution contains the HBT at a concentration of 0.1 mM to 0.5 mM, the violuric acid at a concentration of 0.5 mM to 1 mM, the acetonitrile at a concentration of 10%, and the Tween 80 at a concentration of 1%.

In this specific example, the acetonitrile is added as a cosolvent to the crude enzyme solution, and the Tween 80 is added as a dispersant to the crude enzyme solution.
Specific Example IV: This specific example differs from any one of Specific Examples I to III only in that: the crude enzyme solution of the white rot fungi shows a laccase activity of 10 U/mL.
Specific Example V: This specific example differs from any one of Specific Examples I to IV only in that: the crude enzyme solution of the white rot fungi has a natural pH value.
Specific Example VI: This specific example differs from any one of Specific Examples I to V only in that: the crude enzyme solution of the white rot fungi is prepared by subjecting a Coriolus versicolor strain to culture in a broth at 25° C. to 31° C. and 120 r/min to 160 r/min for 12 d to 18 d, conducting centrifugal separation at 9,000 r/min to 11,000 r/min for 10 mM to obtain a supernatant, and then diluting the supernatant; and 1 L of the broth contains 15.00 g to 30.00 g of bran, 0.40 g to 0.50 g of NH₄Cl, 0.20 g of KH₂PO₄, 0.05 g of MgSO₄·7H₂O, 0.01 g of CaCl₂, 1.00 g of the Tween 80, 1.00 mL of an inorganic solution, and distilled water as a balance.

In an example of the present disclosure, the broth has a natural pH value.

In an example of the present disclosure, 1 L of the inorganic solution contains: 3.00 g of MgSO₄·7H₂O, 0.50 g of MnSO₄·H₂O, 1.00 g of NaCl, 0.10 g of FeSO₄·7H₂O, 0.10 g of CoCl₂, 0.10 g of ZnSO₄·7H₂O, 0.10 g of CuSO₄·5H₂O, 0.01 g of KAl(SO₄)_{2·}12H₂O, 0.01 g of H₃BO₃, 0.01 g of Na₂MoO₄·2H₂O, and distilled water as a balance.

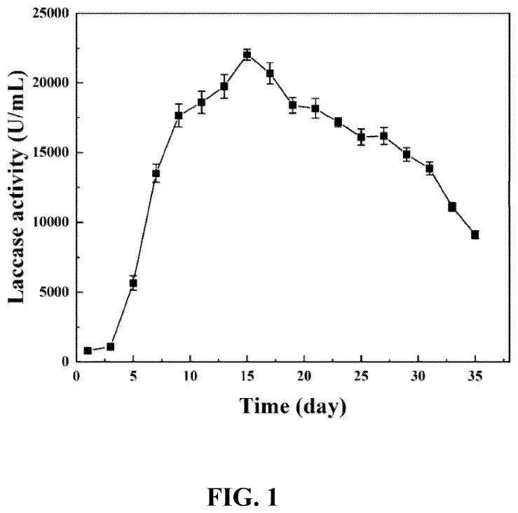

In the present disclosure, Coriolus versicolor is selected. In this specific example, a strain Coriolus versicolor 5.161 is available from the Environmental Microbiology Laboratory of Northeast Forestry University, and has been published in the master's thesis of Lina Du from Northeast Forestry University “Screening and degradation characteristics of white rot fungi strain that efficiently degrades polycyclic aromatic hydrocarbons (PAHs)” in 2010 for public use. FIG. 1 shows the changes of laccase activity in a crude enzyme solution during a process of putting Coriolus versicolor into a broth and then culturing at 28° C. and 135 r/min for 35 d in this specific example. The crude enzyme solution is diluted, and the laccase activity in the crude enzyme solution is diluted to 10 U/mL for the preparation of the immobilized crude enzyme for degrading complex PAHs in soil.
Specific Example VII: The present disclosure provides a preparation method of an immobilized crude enzyme for degrading complex PAHs in soil, including the following steps:
Step 1, washing and drying a chestnut inner shell, and grinding into a powdered material.
Step 2, subjecting the powdered material to pyrolysis in a crucible at 600° C. for 3 h, taking out after cooling, adding with a citric acid solution, and conducting modification by shaking in a shaker at 25° C. to 30° C. and 130 r/min to 180 r/min for 12 h to 48 h to obtain modified biochar.
Step 3, adding the modified biochar to the crude enzyme solution of the white rot fungi according to any one of Specific Examples I to VI, shaking an obtained mixture in a shaker at 25° C. to 30° C. and 130 r/min to 180 r/min for 3 h to 48 h, conducting centrifugation at 5,000 r/min to 10,000 r/min for 5 min to 10 min, and collecting treated biochar to obtain the immobilized crude enzyme for degrading complex PAHs in soil.
Specific Example VIII: The present disclosure provides a preparation method of an immobilized crude enzyme for degrading complex PAHs in soil, including the following steps:
Step 1, washing and drying inside and outside of a chestnut inner shell, and grinding into a powdered material.
Step 2, subjecting the powdered material to pyrolysis in a crucible at 600° C. for 3 h, taking out after cooling, adding with a 2 mM citric acid solution, and conducting modification by shaking in a shaker at 25° C. and 150 r/min for 24 h to obtain modified biochar.
Step 3, adding 1 g of the modified biochar to 5 ml of the crude enzyme solution of the white rot fungi, shaking an obtained mixture in a shaker at 25° C. to 30° C. and 130 r/min to 180 r/min for 24 h, conducting centrifugation at 8,000 r/min for 8 min, and collecting treated biochar to obtain the immobilized crude enzyme for degrading complex PAHs in soil.

The white rot fungi are Coriolus versicolor 5.161, and a crude enzyme solution thereof is prepared by subjecting the Coriolus versicolor 5.161 to culture in a broth at 28° C. and 120 r/min for 15 d, conducting centrifugal separation at 10,000 r/min for 10 min to obtain a supernatant, and then diluting the supernatant; and 1 L of the broth contains 25.00 g of bran, g of NH₄Cl, 0.20 g of KH₂PO₄, 0.05 g of MgSO₄·7H₂O, 0.01 g of CaCl₂), 1.00 g of the Tween 80, 1.00 mL of an inorganic solution, and distilled water as a balance. The broth has a natural pH value. 1 L of the inorganic solution contains: 3.00 g of MgSO₄·7H₂O, 0.50 g of MnSO₄·H₂O, 1.00 g of NaCl, 0.10 g of FeSO₄·7H₂O, 0.10 g of CoCl₂, 0.10 g of ZnSO₄·7H₂O, g of CuSO₄·5H₂O, 0.01 g of KAl(SO₄)₂·12H₂O, 0.01 g of H₃BO₃, 0.01 g of Na₂MoO₄·2H₂O, and distilled water as a balance. The crude enzyme solution further contains a solution of copper ions, acetonitrile, Tween 80, and a mediator; the copper ions in the crude enzyme solution have a concentration of 1.2 mM, and the mediator contains HBT and violuric acid; and the crude enzyme solution has the HBT at a concentration of 0.3 mM, the violuric acid at a concentration of 0.7 mM, the acetonitrile at a concentration of 10%, and the Tween 80 at a concentration of 1%. The crude enzyme solution has a natural pH value. The crude enzyme solution shows a laccase activity of 10 U/mL.

In this specific example, a strain Coriolus versicolor 5.161 is available from the Environmental Microbiology Laboratory of Northeast Forestry University, and has been published in the master's thesis of Lina Du from Northeast Forestry University “Screening and degradation characteristics of white rot fungi strain that efficiently degrades polycyclic aromatic hydrocarbons (PAHs)” in 2010 for public use.
Specific Example IX: The present disclosure provides a preparation method of an immobilized crude enzyme for degrading complex PAHs in soil, including the following steps:
Step 1, washing and drying inside and outside of a chestnut inner shell, and grinding into a powdered material.
Step 2, subjecting the powdered material to pyrolysis in a crucible at 600° C. for 3 h, taking out after cooling, adding with a 2 mM citric acid solution, and conducting modification by shaking in a shaker at 28° C. and 160 r/min for 40 h to obtain modified biochar.
Step 3, adding 1 g of the modified biochar to 5 ml of the crude enzyme solution of the white rot fungi, shaking an obtained mixture in a shaker at 30° C. and 180 r/min for 5 h, conducting centrifugation at 6,000 r/min for 6 mM, and collecting treated biochar to obtain the immobilized crude enzyme for degrading complex PAHs in soil.

The white rot fungi are Coriolus versicolor, and a crude enzyme solution thereof is prepared by subjecting the Coriolus versicolor to culture in a broth at 30° C. and 150 r/min for 18 d, conducting centrifugal separation at 10,000 r/min for 10 mM to obtain a supernatant, and then diluting the supernatant; and 1 L of the broth contains 20.00 g of bran, 0.50 g of NH₄Cl, 0.20 g of KH₂PO₄, 0.05 g of MgSO₄·7H₂O, 0.01 g of CaCl₂), 1.00 g of the Tween 80, 1.00 mL of an inorganic solution, and distilled water as a balance. The broth has a natural pH value. 1 L of the inorganic solution contains: 3.00 g of MgSO₄·7H₂O, 0.50 g of MnSO₄H₂O, 1.00 g of NaCl, 0.10 g of FeSO₄·7H₂O, 0.10 g of CoCl₂, 0.10 g of ZnSO₄·7H₂O, 0.10 g of CuSO₄·5H₂O, 0.01 g of KAl(SO₄)₂·12H₂O, 0.01 g of H₃BO₃, 0.01 g of Na₂MoO₄·2H₂O, and distilled water as a balance. The crude enzyme solution further contains a solution of copper ions, acetonitrile, Tween 80, and a mediator; the copper ions in the crude enzyme solution have a concentration of 1.5 mM, and the mediator contains HBT and violuric acid; and the crude enzyme solution has the HBT at a concentration of 0.4 mM, the violuric acid at a concentration of 0.6 mM, the acetonitrile at a concentration of 10%, and the Tween 80 at a concentration of 1%. The crude enzyme solution has a natural pH value. The crude enzyme solution shows a laccase activity of 10 U/mL.

In this specific example, the selected Coriolus versicolor is available from the Environmental Microbiology Laboratory of Northeast Forestry University, which has been publicly used in “The effect of the substrate phenanthrene on the production of laccase by Coriolus versicolor” in “Modern Agricultural Science and Technology” in the 10th issue of 2013.

### Example 1

A. 50 g of test soil mixed with PAHs was put into a flower pot (a total concentration of PAHs in the test soil was 3.96 mg/L).
B. 1 g each of unmodified chestnut inner shell, unmodified chestnut shell, acid-modified chestnut outer shell biochar, alkali-modified chestnut inner shell biochar, alkali-modified chestnut outer shell biochar, rice husk biochar, and corn straw biochar was separately added to 5 ml of a crude enzyme solution of a Coriolus versicolor strain to prepare corresponding immobilized crude enzyme solutions, respectively.
C. 1 g each of the immobilized crude enzyme for degrading complex PAHs in soil prepared by Specific Example VIII of the present disclosure, the crude enzyme solution of Coriolus versicolor in step B, and the alkali-modified chestnut inner shell biochar-immobilized crude enzyme, the rice husk biochar-immobilized crude enzyme, and the corn straw biochar-immobilized crude enzyme prepared by step B were added to the flower pot mixed with PAHs-containing test soil.
D. An appropriate amount of distilled water was added in the flower pot to make a soil moisture content to be 60%; water was replenished every other day to keep the soil moisture content at 60%.
E. After 10 d, the PAHs in the soil sample were extracted for determination, and a remediation effect of the PAHs-contaminated soil was calculated.

The white rot fungi were Coriolus versicolor, and a crude enzyme solution thereof was prepared by subjecting the Coriolus versicolor 5.161 to culture in a broth at 28° C. and 120 r/min for 15 d, conducting centrifugal separation at 10,000 r/min for 10 mM to obtain a supernatant, and then diluting the supernatant; and 1 L of the broth contained 25.00 g of bran, g of NH₄Cl, 0.20 g of KH₂PO₄, 0.05 g of MgSO₄·7H₂O, 0.01 g of CaCl₂, 1.00 g of Tween 80, 1.00 mL of an inorganic solution, and distilled water as a balance. The broth had a natural pH value. 1 L of the inorganic solution contained: 3.00 g of MgSO₄·7H₂O, 0.50 g of MnSO₄·H₂O, 1.00 g of NaCl, 0.10 g of FeSO₄·7H₂O, 0.10 g of CoCl₂, 0.10 g of ZnSO₄·7H₂O, g of CuSO₄·5H₂O, 0.01 g of KAl(SO₄)₂·12H₂O, 0.01 g of H₃BO₃, 0.01 g of Na₂MoO₄·2H₂O, and distilled water as a balance. The crude enzyme solution further contained a solution of copper ions, acetonitrile, Tween 80, and a mediator; the copper ions in the crude enzyme solution had a concentration of 1.2 mM, and the mediator contained HBT and violuric acid; and the crude enzyme solution had the HBT at a concentration of 0.3 mM, the violuric acid at a concentration of 0.7 mM, the acetonitrile at a concentration of 10%, and the Tween 80 at a concentration of 1%. The crude enzyme solution had a natural pH value. The crude enzyme solution showed a laccase activity of 10 U/mL.

In this specific example, a strain Coriolus versicolor 5.161 came from the Environmental Microbiology Laboratory of Northeast Forestry University, and had been published in the master's thesis of Lina Du from Northeast Forestry University “Screening and degradation characteristics of white rot fungi strain that efficiently degrades polycyclic aromatic hydrocarbons (PAHs)” in 2010 for public use.

The unmodified chestnut inner shell was made by washing the inside and outside of the chestnut inner shell, drying, taking out, and grinding.

The unmodified chestnut outer shell was made by washing the inside and outside of the chestnut outer shell, drying, taking out, and grinding.

The acid-modified chestnut outer shell biochar was a powdered material made by washing the inside and outside of the chestnut shell, drying, taking out, and grinding; the powdered material was subjected to pyrolysis in a crucible at 600° C. for 3 h, taken out after cooling, added with a 2 mM citric acid solution, and modification was conducted by shaking in a shaker at 25° C. and 150 r/min for 24 h to obtain modified biochar.

The alkali-modified chestnut inner shell biochar was a powdered material made by washing the inside and outside of the chestnut inner shell, drying, taking out, and grinding; the powdered material was subjected to pyrolysis in a crucible at 600° C. for 3 h, taken out after cooling, added with a saturated potassium hydroxide solution, and modification was conducted by shaking in a shaker at 25° C. and 150 r/min for 24 h to obtain modified biochar.

The alkali-modified chestnut outer shell biochar was a powdered material made by washing the inside and outside of the chestnut outer shell, drying, taking out, and grinding; the powdered material was subjected to pyrolysis in a crucible at 600° C. for 3 h, taken out after cooling, added with a saturated potassium hydroxide solution, and modification was conducted by shaking in a shaker at 25° C. and 150 r/min for 24 h to obtain modified biochar.

The rice husk biochar was made by washing and drying the rice husk, taking out and grinding.

The corn straw biochar was made by washing and drying the corn straw, taking out and grinding.

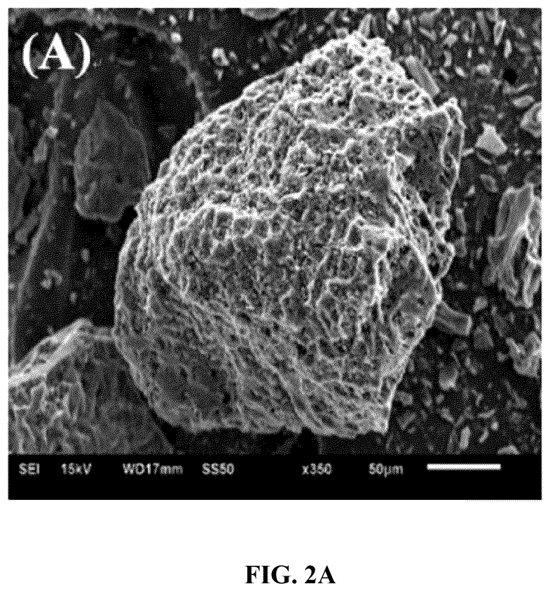

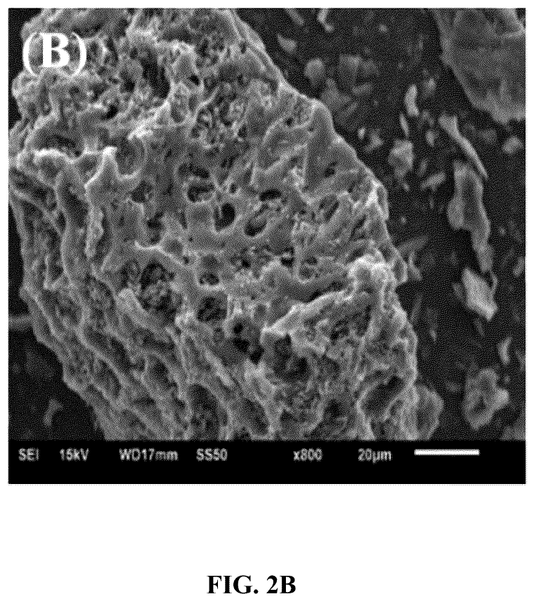

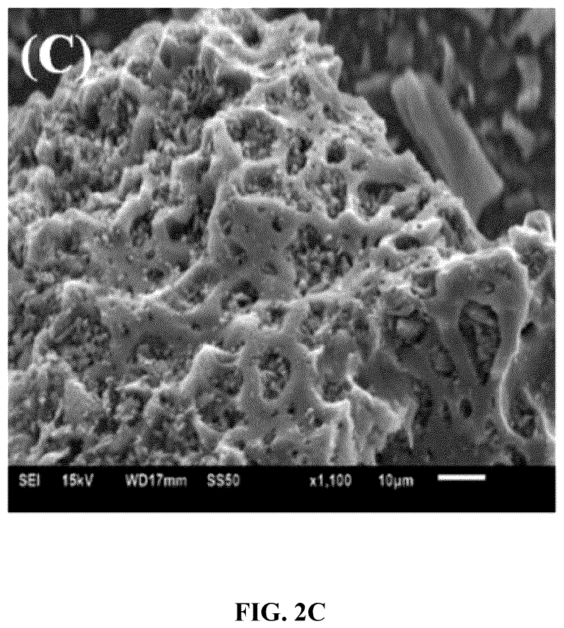

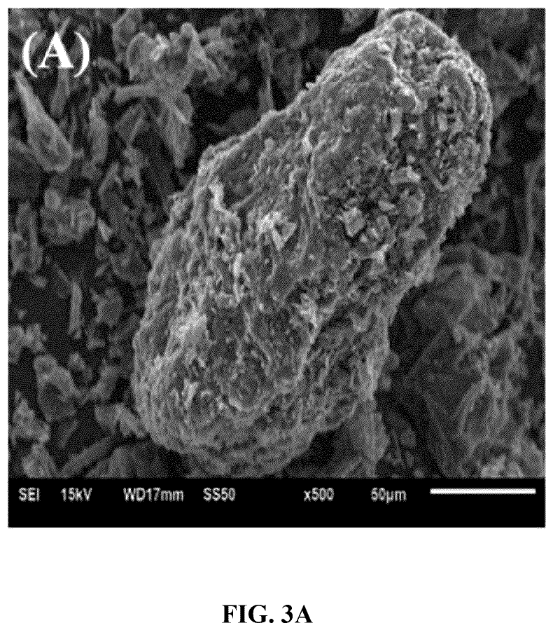

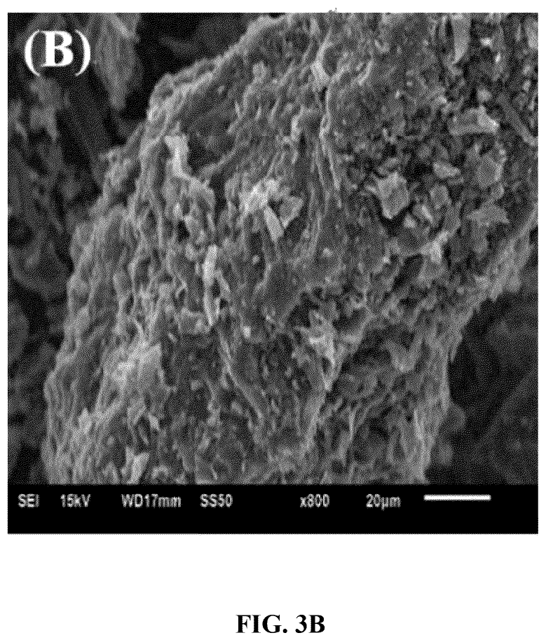

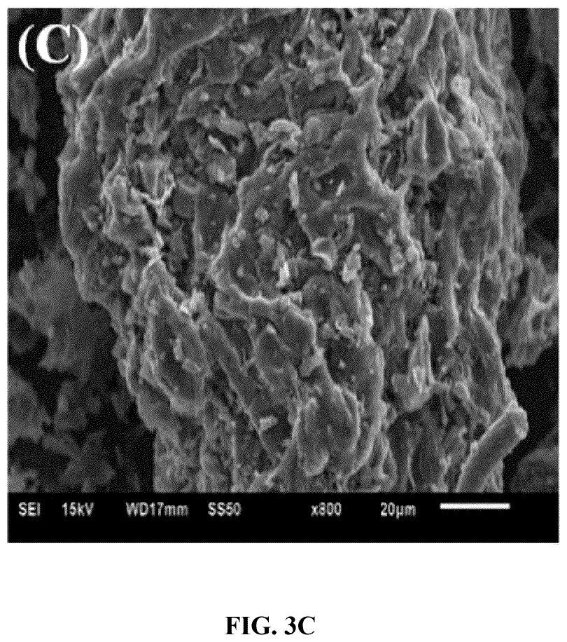

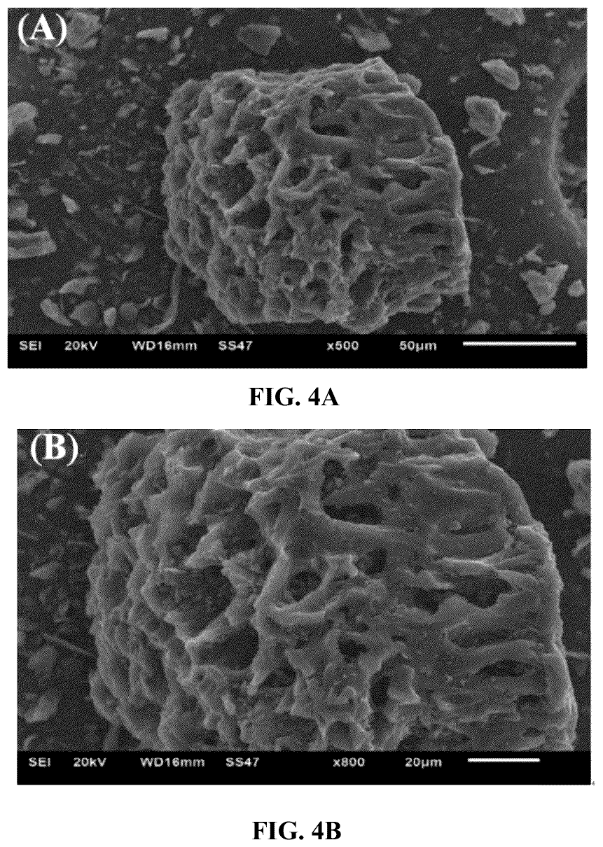

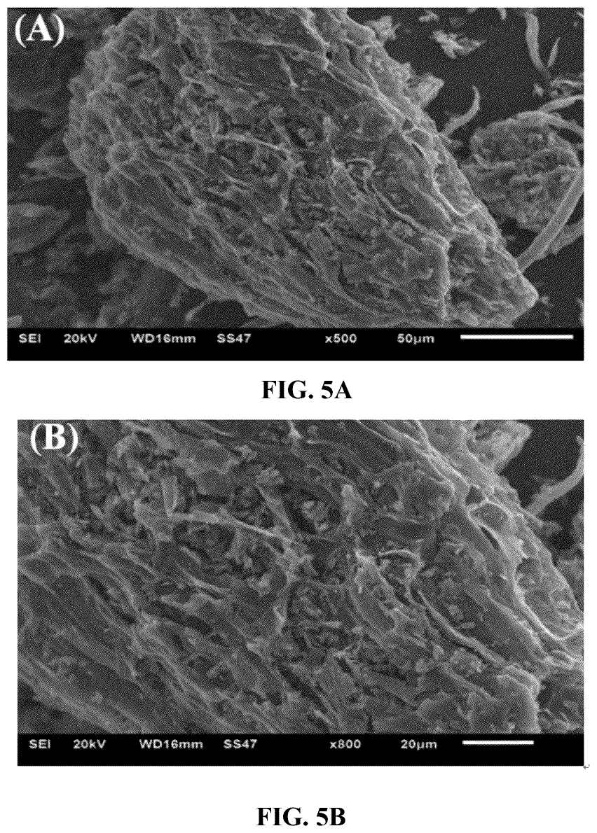

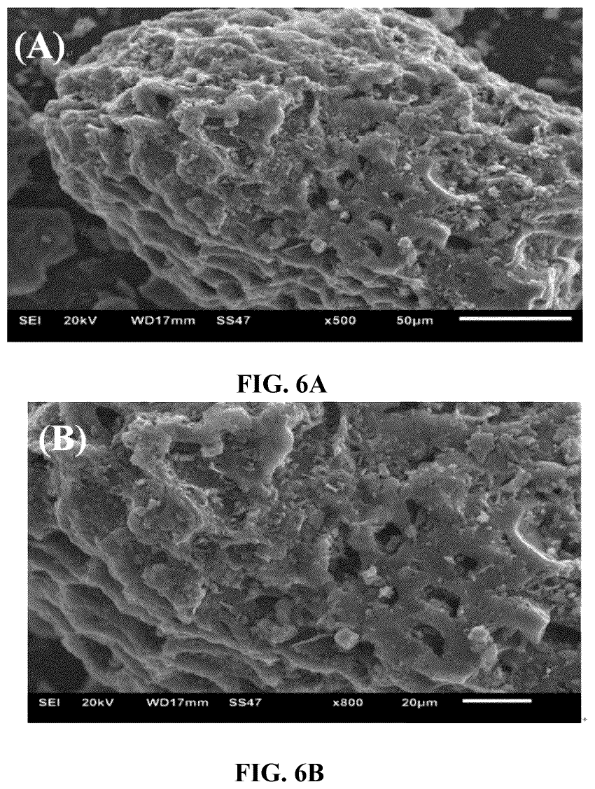

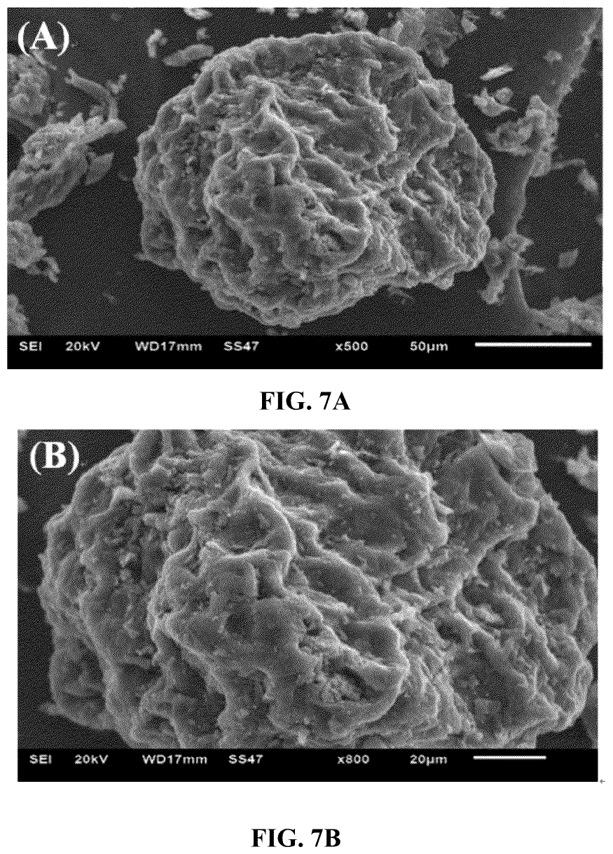

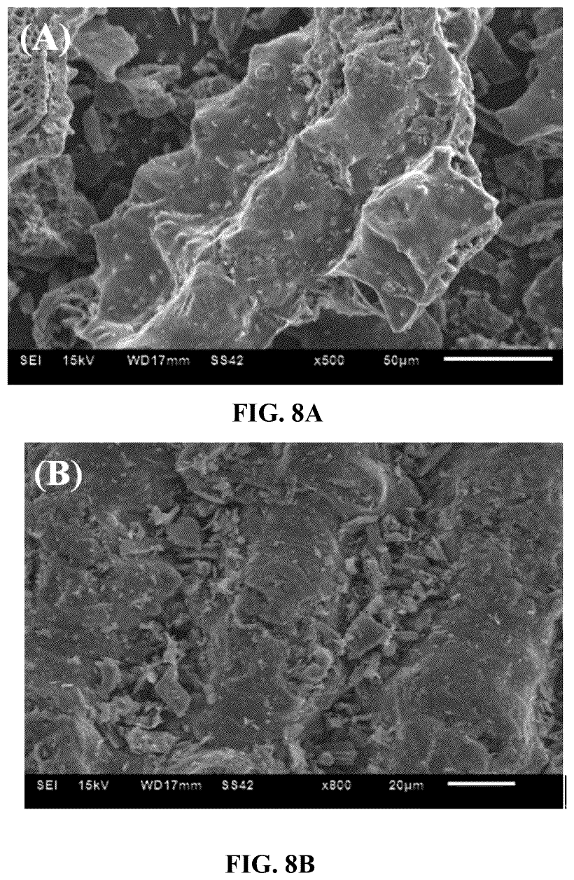

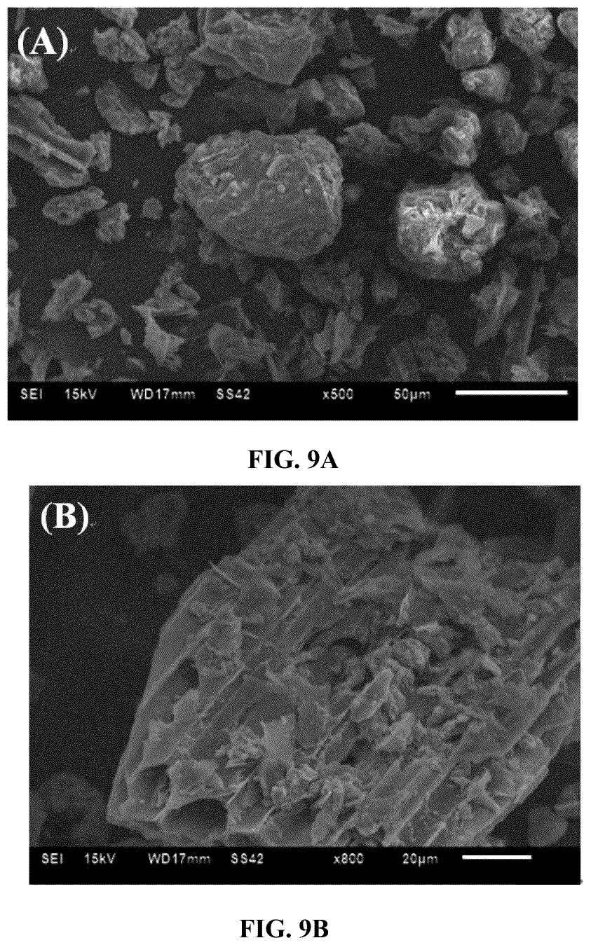

The results were observed by a scanning electron microscope (SEM): the microstructure of the chestnut inner shell biochar in this example was shown in FIGS. 2A-C. The microstructure of chestnut outer shell biochar was shown in FIGS. 3A-C. The microstructure of the modified biochar (acid-modified chestnut inner shell biochar) in the Specific Example VIII was shown in FIGS. 4A-B. The microstructure of the acid-modified chestnut outer shell biochar was shown in FIGS. 5A-B. The microstructure of alkali-modified chestnut inner shell biochar was shown in FIGS. 6A-B. The microstructure of alkali-modified chestnut outer shell biochar was shown in FIGS. 7A-B. The microstructure of rice husk biochar was shown in FIGS. 8A-B. The microstructure of corn straw biochar was shown in FIGS. 9A-B.

After comparing FIGS. 2A-C to FIGS. 9A-B, chestnut inner shell biochar: the unmodified inner shell showed abundant pores and a large specific surface area, but some pores were blocked by fine powder. Inner shell biochar after acid and alkali modification: the channel was clearer, the etching on the surface of the inner shell sample modified by alkali was more obvious, and the pores were clearer. The pores of the acid-modified inner shell were clearer, and there was less residual debris inside the pores, and the etching effect on the surface of the sample was not obvious. Compared with the chestnut inner shell biochar and the alkali-modified chestnut inner shell biochar, the acid-modified chestnut inner shell biochar had a larger specific surface area, which was more conducive to enzyme immobilization. The unmodified chestnut shell showed fewer channels and the channels were blocked seriously. After acid and alkali modification, the channels were clear, and the remaining powder inside became less, indicating that the acid-base modification had improved the bearing capacity of the chestnut outer shell. However, the outer shell had fewer pores than the inner shell and was severely clogged. SEM observation found that there were extremely few samples available in the outer shell samples, and the field of view showed basically a “needle-like and non-porous” sample that could not be used as an immobilization carrier. Compared with the commercially available rice husk biochar and corn straw biochar, the acid-modified chestnut inner shell biochar had a clear pore structure.

The Zeta potentials of the crude enzyme solution of the white rot fungi (Coriolus versicolor) and the eight kinds of biochars were measured five times to obtain an average, and the results were shown in Table 1.

TABLE 1

| Name | Zeta potential (mV) |
|---|---|
| Crude enzyme solution of white rot fungi | −3.584 ± 0.70 |
| Chestnut inner shell biochar | −26.34 ± 0.61 |
| Chestnut outer shell biochar | −29.04 ± 1.35 |
| Acid-modified chestnut inner shell biochar | −13 ± 1.56 |
| Acid-modified chestnut outer shell biochar | −24.36 ± 0.9 |
| Alkali-modified chestnut inner shell biochar | −42.74 ± 2.35 |
| Alkali-modified chestnut outer shell biochar | −44.58 ± 4.07 |
| Corn straw biochar | −31.86 ± 1.11 |
| Rice husk biochar | −46.96 ± 1.32 |

The Zeta potential of biochar was negative, that is, the surface was negatively charged. The acid-modified chestnut inner shell had the highest potential (−13 mV±1.56 mV), which was significantly higher than that of two commercially available biochars (rice husk biochar and corn straw biochar), and was increased by 13.34 mV compared with the Zeta potential of the inner shell before modification. The Zeta potential of the crude enzyme solution of white rot fungi strain was also negative, and the surface was also negatively charged. Therefore, a more positively charged biochar on the surface indicates a smaller the electrostatic repulsion between the biochar and the crude enzyme solution of the white rot fungi strain. The higher the Zeta potential, the more favorable the adsorption of microorganisms. Some of the original indigenous microorganisms in the polluted soil have a certain ability to degrade PAHs. However, due to the hydrophobicity of PAHs and the limitation of microbial living environment, it is difficult for these indigenous microorganisms to degrade PAHs. The addition of appropriate biochar can promote the degradation potential of some indigenous microorganisms to PAHs. Therefore, the acid-modified chestnut inner shell biochar with a higher Zeta potential had the strongest binding ability to the crude enzyme solution of white rot fungi strains, as well as a certain promotion ability to degrade PAHs by some indigenous microorganisms with degradation potential. Meanwhile, combined with the SEM images of the eight biochars (FIGS. 2A-C to FIGS. 9A-B), it can be seen that the acid-modified chestnut inner shell biochar had a more obvious pore structure.

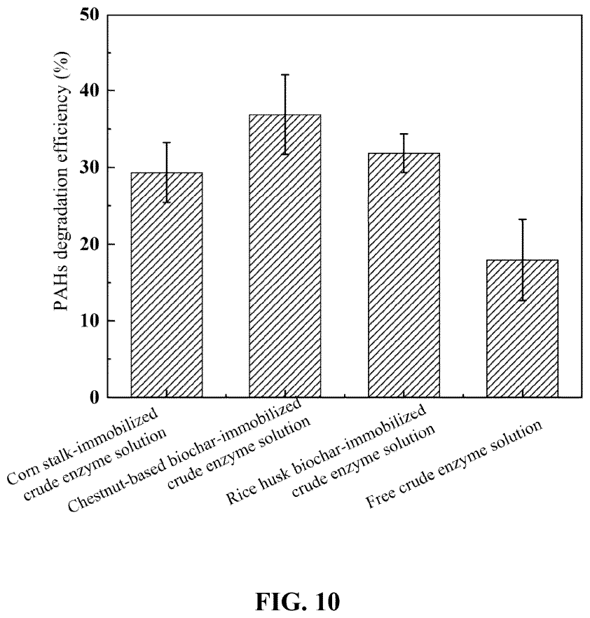

The four remediation materials had certain degradability to PAHs-contaminated soil. Compared with the free laccase (that is, the crude enzyme solution of the white rot fungi strain), the degradation effects of the three immobilized crude enzymes were significantly improved. Under the same conditions, the loaded immobilized crude enzyme degraded a larger amount of PAHs in the soil, showing a better degradation effect. In addition, the acid-modified chestnut inner shell biochar-loaded immobilized crude enzyme could achieve a remediation effect of 36.87% within a remediation period of 10 d. Compared with two commercially available biochars (rice husk biochar and corn straw biochar) loaded with immobilized crude enzymes, the effect was significantly improved, as shown in FIG. 10. The acid-modified chestnut inner shell biochar had a more obvious pore structure, and was related to the smaller electrostatic repulsion of the crude enzyme solution of white rot fungi (Coriolus versicolor) strains. Both the loaded immobilized crude enzyme and the free crude enzyme solution had a certain degradation effect on PAHs. The immobilized crude enzyme for degrading complex PAHs in soil showed better activity, higher stability, and greater catalytic degradation effect on PAHs. The free crude enzyme solution is quickly inactivated due to the complex environmental factors of the soil, while the loaded immobilized crude enzyme is attached to the pores of biochar, thus largely avoiding the influence of the complex soil environment. Even in complex polluted soils, the immobilized crude enzyme for degrading complex PAHs in soil of the present disclosure has a certain effect on soil restoration. This shows that the immobilized crude enzyme for degrading complex PAHs in soil of the present disclosure has broad application prospects in the bioremediation of PAHs-contaminated soil in the future.

What is claimed is:

1. An immobilized crude enzyme for degrading complex polycyclic aromatic hydrocarbons (PAHs) in soil, wherein a solution of the immobilized crude enzyme is prepared from an acid-modified chestnut inner shell and a crude enzyme solution of white rot fungi; and there are copper ions in the crude enzyme solution of the white rot fungi.

2. The immobilized crude enzyme for degrading complex PAHs in soil according to claim 1, wherein a concentration of the copper ions in the crude enzyme solution of the white rot fungi is 1 mM to 2 mM.

3. The immobilized crude enzyme for degrading complex PAHs in soil according to claim 1, wherein the crude enzyme solution of the white rot fungi further comprises acetonitrile, Tween 80, and a mediator; the mediator comprises 1-hydroxybenzotriazole (HBT) and violuric acid; and the crude enzyme solution has the HBT at a concentration of 0.1 mM to 0.5 mM, the violuric acid at a concentration of 0.5 mM to 1 mM, the acetonitrile at a concentration of 10%, and the Tween 80 at a concentration of 1%.

4. The immobilized crude enzyme for degrading complex PAHs in soil according to claim 2, wherein the crude enzyme solution of the white rot fungi further comprises acetonitrile, Tween 80, and a mediator; the mediator comprises 1-hydroxybenzotriazole (HBT) and violuric acid; and the crude enzyme solution has the HBT at a concentration of 0.1 mM to 0.5 mM, the violuric acid at a concentration of 0.5 mM to 1 mM, the acetonitrile at a concentration of 10%, and the Tween 80 at a concentration of 1%.

5. The immobilized crude enzyme for degrading complex PAHs in soil according to claim 3, wherein the crude enzyme solution of the white rot fungi shows a laccase activity of 10 U/mL.

6. The immobilized crude enzyme for degrading complex PAHs in soil according to claim 4, wherein the crude enzyme solution of the white rot fungi shows a laccase activity of 10 U/mL.

7. The immobilized crude enzyme for degrading complex PAHs in soil according to claim 3, wherein the crude enzyme solution of the white rot fungi has a natural pH value.

8. The immobilized crude enzyme for degrading complex PAHs in soil according to claim 4, wherein the crude enzyme solution of the white rot fungi has a natural pH value.

9. The immobilized crude enzyme for degrading complex PAHs in soil according to claim 1, wherein the white rot fungi is Coriolus versicolor.

10. The immobilized crude enzyme for degrading complex PAHs in soil according to claim 3, wherein the crude enzyme solution of the white rot fungi is prepared by subjecting a Coriolus versicolor strain to culture in a broth at 25° C. to 31° C. and 120 r/min to 160 r/min for 12 d to 18 d, conducting centrifugal separation at 9,000 r/min to 11,000 r/min for 10 min to obtain a supernatant, and then diluting the supernatant; and 1 L of the broth comprises 15.00 g to 30.00 g of bran, 0.40 g to 0.50 g of NH₄Cl, 0.20 g of KH₂PO₄, 0.05 g of MgSO₄·7H₂O, 0.01 g of CaCl₂, 1.00 g of the Tween 80, 1.00 mL of an inorganic solution, and distilled water as a balance.

11. The immobilized crude enzyme for degrading complex PAHs in soil according to claim 4, wherein the crude enzyme solution of the white rot fungi is prepared by subjecting a Coriolus versicolor strain to culture in a broth at 25° C. to 31° C. and 120 r/min to 160 r/min for 12 d to 18 d, conducting centrifugal separation at 9,000 r/min to 11,000 r/min for 10 min to obtain a supernatant, and then diluting the supernatant; and 1 L of the broth comprises 15.00 g to 30.00 g of bran, 0.40 g to 0.50 g of NH₄Cl, 0.20 g of KH₂PO₄, 0.05 g of MgSO₄·7H₂O, 0.01 g of CaCl₂, 1.00 g of the Tween 80, 1.00 mL of an inorganic solution, and distilled water as a balance.

12. A preparation method of an immobilized crude enzyme for degrading complex PAHs in soil, comprising the following steps:
step 1, washing and drying a chestnut inner shell, and grinding into a powdered material;
step 2, subjecting the powdered material to pyrolysis in a crucible at 600° C. for 3 h, taking out after cooling, adding with a citric acid solution, and conducting modification by shaking in a shaker at 25° C. to 30° C. and 130 r/min to 180 r/min for 12 h to 48 h to obtain modified biochar; and
step 3, adding the modified biochar to the crude enzyme solution of the white rot fungi according to claim 1, shaking an obtained mixture in a shaker at 25° C. to 30° C. and 130 r/min to 180 r/min for 3 h to 48 h, conducting centrifugation at 5,000 r/min to 10,000 r/min for 5 min to 10 mM, and collecting treated biochar to obtain the immobilized crude enzyme for degrading complex PAHs in soil.

13. The preparation method according to claim 12, wherein a concentration of the copper ions in the crude enzyme solution of the white rot fungi is 1 mM to 2 mM.

14. The preparation method according to claim 12, wherein the crude enzyme solution of the white rot fungi further comprises acetonitrile, Tween 80, and a mediator; the mediator comprises 1-hydroxybenzotriazole (HBT) and violuric acid; and the crude enzyme solution has the HBT at a concentration of 0.1 mM to 0.5 mM, the violuric acid at a concentration of 0.5 mM to 1 mM, the acetonitrile at a concentration of 10%, and the Tween 80 at a concentration of 1%.

15. The preparation method according to claim 13, wherein the crude enzyme solution of the white rot fungi further comprises acetonitrile, Tween 80, and a mediator; the mediator comprises 1-hydroxybenzotriazole (HBT) and violuric acid; and the crude enzyme solution has the HBT at a concentration of 0.1 mM to 0.5 mM, the violuric acid at a concentration of 0.5 mM to 1 mM, the acetonitrile at a concentration of 10%, and the Tween 80 at a concentration of 1%.

16. The preparation method according to claim 14, wherein the crude enzyme solution of the white rot fungi shows a laccase activity of 10 U/mL.

17. The preparation method according to claim 15, wherein the crude enzyme solution of the white rot fungi shows a laccase activity of 10 U/mL.

18. The preparation method according to claim 14, wherein the crude enzyme solution of the white rot fungi has a natural pH value.

19. The preparation method according to claim 12, wherein the white rot fungi is Coriolus versicolor.

20. The preparation method according to claim 14, wherein the crude enzyme solution of the white rot fungi is prepared by subjecting a Coriolus versicolor strain to culture in a broth at 25° C. to 31° C. and 120 r/min to 160 r/min for 12 d to 18 d, conducting centrifugal separation at 9,000 r/min to 11,000 r/min for 10 min to obtain a supernatant, and then diluting the supernatant; and 1 L of the broth comprises 15.00 g to 30.00 g of bran, 0.40 g to 0.50 g of NH₄Cl, 0.20 g of KH₂PO₄, 0.05 g of MgSO₄·7H₂O, 0.01 g of CaCl₂, 1.00 g of the Tween 80, 1.00 mL of an inorganic solution, and distilled water as a balance.
